(19) United States
(12) Patent Application Publication
(10) Pub. No.: US 2023/0340603 A1
(43) Pub. Date: Oct. 26, 2023

(54) METHODS FOR CLASSIFICATION OF LIVER DISEASE

(71) Applicant: TENSOR BIOSCIENCES, INC., Palo Alto, CA (US)

(72) Inventors: Leila Celeste SIDOW, Palo Alto, CA (US); Arend SIDOW, Palo Alto, CA (US); Anton VALOUEV, Palo Alto, CA (US); Aijaz AHMED, Los Altos, CA (US)

(21) Appl. No.: 18/344,616

(22) Filed: Jun. 29, 2023

Related U.S. Application Data
(63) Continuation of application No. 18/254,433, filed on Jan. 1, 0001.
(60) Provisional application No. 63/120,043, filed on Dec. 1, 2020.
(60) Provisional application No. 63/153,032, filed on Feb. 24, 2021.

(51) Int. Cl. C12Q 1/6883

(52) U.S. Cl. CPC C12Q 1/6883; C12Q 2600/154; C12Q 2600/118

(57) ABSTRACT

A method of classifying a liver disease by analyzing a DNA sample, wherein the DNA sample comprises cfDNA and/or blood cell DNA, the method comprising: obtaining the DNA sample; determining CpG methylation status at CpG sites of DNA molecules of the DNA sample; identifying a methylation pattern based on the CpG methylation status of the DNA molecules; assigning to the sample a liver disease classification based on the methylation pattern.

### 1 RELATED APPLICATIONS

This application is a continuation application of U.S. application Ser. No. 18/254,433, filed May 25, 2023, which is a 371 application of International Application No. PCT/US2021/061244, filed Nov. 30, 2021, which claims benefit of U.S. provisional patent application No. 63/120,043, filed on Dec. 1, 2020, and U.S. provisional patent application No. 63/153,032, filed on Feb. 24, 2021, each of which are incorporated herein by reference in their entirety.

### 2 FIELD OF THE INVENTION

The invention relates to methods of developing methylation analyses for disease conditions, such as liver diseases, as well as methods for conducting such analyses, and methods of selecting treatment for, and treating, such disease conditions.

### 3 BACKGROUND OF THE INVENTION

Non-alcoholic fatty liver disease (NAFLD) is the most prevalent form of chronic liver disease. NAFLD often progresses to nonalcoholic steatohepatitis (NASH), which can progress to cirrhosis, and eventually progress to liver cancer. The symptoms of these disease stages tend to lie on a continuum, starting with fatigue and abdominal pain as in some NAFLD cases. The same symptoms also tend to be common with NASH, with severe NASH cases presenting symptoms of cirrhosis and liver failure. Because of these similarities, limited options exist for accurate diagnosis and staging of these conditions. Often, diagnosis involves a liver biopsy, a risky procedure. Attempts at developing non-invasive modalities for diagnosis and staging have been only partly effective. There is a need in the art for a robust means for diagnosing and staging liver diseases without requiring liver biopsy.

### 4 SUMMARY

The invention relates to a method of classifying disease conditions by analyzing a DNA sample. The DNA sample may, for example, be a cfDNA sample.

In one embodiment, the method includes classifying a liver disease by analyzing a DNA sample, wherein the DNA sample comprises cfDNA and/or blood cell DNA. The method involves determining CpG methylation status at CpG sites of DNA molecules in the DNA samples obtained, identifying a methylation pattern based on the CpG methylation status of the DNA molecules and assigning to the sample a liver disease classification, based on the methylation pattern.

In another embodiment, the method includes classifying a liver disease by analyzing a DNA sample, wherein the DNA sample comprises cfDNA fragments and/or DNA fragments from blood cells, and the fragments are enriched by hybridization to a set of probes of a targeted panel, using PCR with a panel of primers.

In another embodiment, the method includes classifying a liver disease by analyzing a DNA sample, involving determining CpG methylation status at CpG sites of DNA molecules in the DNA samples obtained; wherein the methylation pattern is used to calculate a methylation level indicating a probability that the sample belongs to a particular liver disease classification.

In another embodiment, the method for classifying a liver disease includes the use of methylation patterns to calculate the methylation level, wherein the methylation level is compared to a cut-off, to classify the liver disease and report the probability of a stage of liver disease, with a score derived from the methylation level of the DNA sample.

In another embodiment, the method involves reporting the probability of a stage of liver disease with a score derived from the methylation level of the DNA sample and classifying the sample as having a probability of no liver disease, non-alcoholic fatty liver disease, non-alcoholic steatohepatitis, liver cirrhosis, and/or liver carcinoma.

In another embodiment, the method involves classifying the sample for a stage of fibrosis, by classifying the sample as having a probability of no fibrosis; portal fibrosis without septa; portal fibrosis with few septa; periportal fibrosis; bridging fibrosis; and/or cirrhosis.

In another embodiment, the method involves classifying the sample for a hepatitis, comprising classifying the sample as having a probability of no hepatitis; non-specific reactive hepatitis; granulomatous hepatitis; chronic active hepatitis; acute hepatitis; autoimmune hepatitis; alcoholic hepatitis; and/or nonalcoholic hepatitis.

In another embodiment, the method involves classifying the sample for a grade of liver inflammation by classifying the sample as having a probability of no inflammation; mild inflammation; moderate inflammation; and/or marked or severe inflammation.

In another embodiment, the method involves classifying the sample for a grade of liver necrosis by classifying the sample as having a probability of no necrosis; mild necrosis; moderate necrosis; and/or marked or severe necrosis.

In another embodiment, the method involves classifying the sample for a level of fat in the liver.

In another embodiment, the methylation pattern used to calculate a methylation level to indicate a probability that the sample belongs to a particular liver disease classification, is established by identifying coefficients for one or more CpG features, by fitting a model based on methylation patterns in the DNA samples from a training set; wherein the samples comprise DNA samples from subjects with or without liver disease.

In another embodiment, the methylation pattern used to calculate a methylation level to indicate a probability that the sample belongs to a particular liver disease classification, is established by identifying coefficients for one or more CpG features, and comprises a single CpG site, a set of CpG sites located on the same DNA fragment, CpG features derived using mutual information analysis or CpG features are derived using L1 logistic regression

In another embodiment, the methylation level may be established by identifying coefficients for one or more CpG features by fitting a model, including but not limited to a logistic regression model with L2 penalty, a logistic regression model with L1 penalty, random forest, neural network, a support vector machine, a gradient boosting algorithm, or a naive Bayes.

In one embodiment, a cfDNA sample comprises genomic regions that are enriched by a targeted panel, wherein the panel is established by a method of selecting a set of genomic regions based on cfDNA samples from subjects with and without liver disease using, mutual information; variation based on a cutoff requirement; or L1 logistic regression

In one embodiment, the targeted panel is established by a method of selecting a set of genomic regions based on liver tissue DNA samples from subjects with and without liver disease using, mutual information; variation based on a cutoff requirement; or L1 logistic regression;

In one embodiment, the targeted panel is established by a method of selecting a set of genomic regions based on samples of DNA obtained from purified hepatocytes, adipocytes, fibroblasts, and/or immune cells using: mutual information; variation based on a cutoff requirement; or L1 logistic regression.

In one embodiment, a DNA sample is blood cell DNA with genomic regions that are enriched by a targeted panel, which is established by a method comprising selecting a set of genomic regions based on blood cell samples from a training set from subjects with and without liver disease using mutual information; variation based on a cutoff requirement; or L1 logistic regression.

In one embodiment, the targeted panel is established by a method of selecting a set of genomic regions based on samples from purified T cells, B cells, granulocytes and/or neutrophils using mutual information; variation based on a cutoff requirement; or L1 logistic regression.

In one embodiment, the method includes classifying a liver disease by analyzing a DNA sample; the method involves determining CpG methylation status at CpG sites of DNA molecules in the DNA samples obtained, by determining the presence of 5mC or 5hmC modifications at individual sites of the DNA molecules using a method comprising methylation-aware sequencing.

In one embodiment, the method includes classifying a liver disease by analyzing a DNA sample; the method involves determining CpG methylation status at CpG sites of DNA molecules in the DNA samples obtained, by determining the average levels of 5mC or 5hmC across individual genomic CpG sites of the DNA molecules using a method comprising a methylation-aware DNA array method.

In one embodiment, the method includes classifying a liver disease by analyzing a DNA sample; the method involves determining CpG methylation status at CpG sites of DNA molecules in the DNA samples obtained, by average levels of 5mC or 5hmC at a selected set of genomic CpG sites of the DNA molecules using a method comprising methylation-aware PCR, qPCR or digital PCR.

In one embodiment, the method involves determining CpG methylation status at CpG sites of DNA molecules in the DNA samples obtained, may include converting the DNA molecules using sodium bisulfite treatment, TET2-assisted DNA oxidation and APOBEC-assisted cytosine deamination.

In one embodiment, the method involves binding the DNA molecules to a DNA array and enriching the sample using probes from the targeted panel performing methylation-aware sequencing of the DNA molecules

In one embodiment, the method involves detecting methylation levels of CpG sites of the DNA molecules using a DNA array, PCR, qPCR or digital PCR.

### 5 BRIEF DESCRIPTION OF THE DRAWINGS

FIG. 1 illustrates a heat map of CpG methylation markers across the different tissue samples; showing each of the selected CpGs (rows) Beta-value per sample (columns). K=7000 variation-based features selected, followed by r=10 rounds of L1 feature selection with C=0.5 yielded 313 liver-specific CpGs.

FIG. 2A illustrates each of the 19 liver-specific CpG's (rows) Beta-value per sample (columns). K=7000 variation-based features selected, followed by r=1 rounds of L1 feature selection with C=0.5.

FIG. 2B illustrates a selection of 19 highly predictive CpGs, their coordinates, and their L2 coefficients for liver tissue.

FIG. 2C illustrates classifying a sample as a liver sample by a logistic regression model with leave-one-out cross validation, using only methylation data from the set of 19 liver-specific CpGs.

FIG. 3A illustrates a selection of 15 highly predictive CpG markers, listed by their cgID with coordinates and L2 coefficients, for distinguishing samples with NAFLD when compared to healthy samples of primary liver tissue.

FIG. 3B illustrates classifying a sample as a NAFLD liver sample by a logistic regression model with leave-one-out cross validation, using only methylation data from the set of 15 NAFLD-specific CpGs.

FIG. 4A illustrates a selection of 16 highly predictive CpG markers, listed by their cgID with coordinates and L2 coefficients, for distinguishing samples with NASH when compared to healthy samples of primary liver tissue.

FIG. 4B illustrates classifying a sample as a NASH liver sample by a logistic regression model with leave-one-out cross validation, using only methylation data from the set of 16 NASH-specific CpGs.

FIG. 5A illustrates a selection of 20 predictive CpG markers, listed by their cgID with coordinates and L2 coefficients, for distinguishing samples with cirrhosis when compared to healthy samples of primary liver tissue.

FIG. 5B illustrates classifying a sample as a cirrhotic liver sample by a logistic regression model with leave-one-out cross validation, using only methylation data from the set of 20 cirrhosis-specific CpGs.

FIG. 6A illustrates a selection of 11 highly predictive CpG markers, listed by their cgID with coordinates and L2 coefficients, for distinguishing samples with cirrhosis from NAFLD primary liver tissue samples.

FIG. 6B illustrates classifying a sample as a cirrhotic liver sample by a logistic regression model with leave-one-out cross validation, using only methylation data from the set of 11 CpGs.

FIG. 7A illustrates a selection of 12 highly predictive CpG markers, listed by their cgID with coordinates and L2 coefficients, for distinguishing samples with cirrhosis from NASH primary liver tissue samples.

FIG. 7B illustrates classifying a sample as a cirrhotic liver sample by a logistic regression model with leave-one-out cross validation, using only methylation data from the set of 12 CpGs.

FIG. 8A illustrates a selection of 13 CpG markers, listed by their cgID with coordinates and L2 coefficients, used for distinguishing between NAFLD and NASH primary liver samples.

FIG. 8B illustrates classifying a sample as a NASH liver sample by a logistic regression model with leave-one-out cross validation, using only methylation data from the set of 13 CpGs.

FIG. 9A illustrates a selection of 12 highly predictive CpG markers, listed by their cgID with coordinates and L2 coefficients, used for distinguishing samples with cirrhosis from healthy cfDNA samples.

FIG. 9B illustrates classifying a sample as a cirrhotic sample by a logistic regression model with leave-one-out cross validation, using only methylation data from the set of 12 CpGs.

FIG. 10A illustrates a selection of 21 CpG markers, listed by their cgID with coordinates and L2 coefficients, used for distinguishing samples with only cirrhosis from samples with cirrhosis and hepatocellular carcinoma

FIG. 10B illustrates classifying a sample as a cirrhotic sample by a logistic regression model with leave-one-out cross validation, using only methylation data from the set of 21 CpGs.

FIG. 11 illustrates the probability that a sample would be classified as a NAFLD Grade 0 liver sample by a logistic regression model using leave-one-out feature selection and cross-validation using only methylation data.

### 6 DETAILED DESCRIPTION

The invention provides methods of classifying a liver disease. The method includes analyzing a DNA sample. The DNA sample may include cfDNA and/or blood cell DNA.

In one aspect, the method includes obtaining the DNA sample; determining CpG methylation status at CpG sites of DNA molecules of the DNA sample; identifying a methylation pattern based on the CpG methylation status of the DNA molecules; and assigning to the sample a liver disease classification based on the methylation pattern.

The DNA sample may include cfDNA fragments. The DNA sample may include DNA fragments from blood cells.

The fragments may be enriched, e.g., by hybridization to a set of probes of a targeted panel or using PCR with a panel of primers.

The methylation pattern may be used to calculate a methylation level. The methylation level may indicate a probability that the sample belongs to a particular liver disease classification.

The invention may also include reporting a probability of a stage of liver disease with a score derived from the methylation level of the DNA sample.

Assigning to the sample a liver disease classification based on the methylation pattern may include comparing the methylation level to a cut-off to classify the liver disease.

Assigning to the sample a liver disease classification based on the methylation pattern may include classifying the sample as having a probability of no liver disease; non-alcoholic fatty liver disease; non-alcoholic steatohepatitis; liver cirrhosis; and/or liver carcinoma.

Assigning to the sample a liver disease classification based on the methylation pattern may include classifying the sample for a stage of fibrosis. Classifying the sample for a stage of fibrosis may include classifying the sample as having a probability of no fibrosis; portal fibrosis without septa; portal fibrosis with few septa; periportal fibrosis; bridging fibrosis; and/or cirrhosis.

Assigning to the sample a liver disease classification based on the methylation pattern may include classifying the sample for a hepatitis. Classifying the sample for a hepatitis may include classifying the sample as having a probability of no hepatitis; non-specific reactive hepatitis; granulomatous hepatitis; chronic active hepatitis; acute hepatitis; autoimmune hepatitis; alcoholic hepatitis; and/or non-alcoholic hepatitis.

Assigning to the sample a liver disease classification based on the methylation pattern may include classifying the sample for a grade of liver inflammation. Classifying the sample for a grade of liver inflammation may include classifying the sample as having a probability of no inflammation; mild inflammation; moderate inflammation; and/or marked or severe inflammation.

Assigning to the sample a liver disease classification based on the methylation pattern may include classifying the sample for a grade of liver necrosis. Classifying the sample for a grade of liver necrosis may include classifying the sample as having a probability of no necrosis; mild necrosis; moderate necrosis; and/or marked or severe necrosis.

Assigning to the sample a liver disease classification based on the methylation pattern may include classifying the sample for a level of fat in the liver.

The methylation level may be established by identifying coefficients for one or more CpG features by fitting a model based on methylation patterns in the DNA sample. The model may be fitted using data from samples from a training set. The samples may include DNA samples from subjects with liver disease; and subjects without liver disease. The training set may also include other data, such as imaging data, medical assessment data, physical signs and symptoms, data corresponding to other analytes such as protein or peptide analytes or metabolic analytes, and any combinations of the foregoing.

The CpG features may include a single CpG site. The CpG features may include a set of CpG sites located on the same DNA fragment. The CpG features may be derived using mutual information analysis. The CpG features may be derived using L1 logistic regression.

The model may include a logistic regression model. The model may include a logistic regression model with L2 penalty. The model may include a logistic regression model with L1 penalty. The model may include a random forest. The model may include a neural network. The model may include a support vector machine. The model may include a gradient boosting algorithm. The model may include a naive Bayes.

The cfDNA sample may include genomic regions that are enriched by a targeted panel.

The targeted panel may be established by a method including selecting a set of genomic regions based on cfDNA samples from subjects with and without liver disease. The selection may be accomplished using mutual information; variation based on a cutoff requirement; and/or L1 logistic regression.

The targeted panel may be established by a method including selecting a set of genomic regions based on liver tissue DNA samples from subjects with and without liver disease. The selection may be accomplished using mutual information; variation based on a cutoff requirement; or L1 logistic regression.

The targeted panel may be established by a method including selecting a set of genomic regions based on samples of DNA obtained from purified hepatocytes, adipocytes, fibroblasts, and/or immune cells. The selection may be accomplished using mutual information; variation based on a cutoff requirement; or L1 logistic regression.

The DNA sample may include blood cell DNA. DNA from the blood cell sample may include genomic regions that are enriched by a targeted panel.

The targeted panel may be established by a method including selecting a set of genomic regions based on blood cell samples from a training set from subjects with and without liver disease. Selection may be accomplished using mutual information; variation based on a cutoff requirement; or L1 logistic regression.

The targeted panel may be established by a method including selecting a set of genomic regions based on samples from purified T cells, B cells, granulocytes and/or neutrophils. Selection may be accomplished using mutual information; variation based on a cutoff requirement; or L1 logistic regression.

Determining CpG methylation status at CpG sites of DNA molecules of the DNA sample may include determining presence of 5mC or 5hmC modifications at individual sites of the DNA molecules using a method including methylation-aware sequencing.

Determining CpG methylation status at CpG sites of DNA molecules of the DNA sample may include determining average levels of 5mC or 5hmC across individual genomic CpG sites of the DNA molecules using a method including a methylation-aware DNA array method.

Determining CpG methylation status at CpG sites of DNA molecules of the DNA sample may include determining average levels of 5mC or 5hmC at a selected set of genomic CpG sites of the DNA molecules using a method including methylation-aware PCR, qPCR or digital PCR.

Determining CpG methylation status at CpG sites of DNA molecules of the DNA sample may include converting the DNA molecules using sodium bisulfite treatment.

Determining CpG methylation status at CpG sites of DNA molecules of the DNA sample may include converting the DNA molecules by TET2-assisted DNA oxidation and APOBEC-assisted cytosine deamination.

Determining CpG methylation status at CpG sites of DNA molecules of the DNA sample may include binding the DNA molecules to a DNA array and enriching the sample using probes from the targeted panel.

Determining CpG methylation status at CpG sites of DNA molecules of the DNA sample may include performing methylation-aware sequencing of the DNA molecules.

Determining CpG methylation status at CpG sites of DNA molecules of the DNA sample may include detecting methylation levels of CpG sites of the DNA molecules using a DNA array.

Determining CpG methylation status at CpG sites of DNA molecules of the DNA sample may include detecting methylation levels of CpG sites of the DNA molecules using PCR, qPCR or digital PCR.

The methods may include a step of obtaining a sample from a subject. The subject may be a human subject.

The methods may include amplifying the targeted panel from the sample using the primers.

The methods may include capturing the DNA molecules from the subject's sample with the targeted panel using the targeted panel probes. In some embodiments, the probes are part of an array. In certain embodiments, the methods of invention include sequencing the targeted panel from the sample.

The methods may include a method of diagnosing or staging a liver condition. For example, the condition may be selected from the group consisting of NASH, NAFLD, fibrosis, and cirrhosis.

The methods may include conducting methylation-aware sequencing of a subset of the cfDNA sample. For example, the subset may include a targeted panel of CpG markers predictive of the diagnosis or staging of a liver condition selected from the group consisting of NASH, NAFLD, and cirrhosis, thereby producing a dataset of methylation status of the predictive CpG markers. The method may include calculating based on a set of predetermined coefficients the diagnosis or staging of the liver condition.

The methods may include a method of analyzing features of the targeted panel from the subject to distinguish between a healthy state and a cirrhosis positive state. In some cases, the targeted panel includes 5, 6, 7, 8, 9, 10, 11, 12, 13 more CpG markers selected from the following cgIDs: cg13851870, cg15476885, cg16646879, cg17189020, cg17373656, cg17479131, cg18048953, cg20149170, cg25009327, cg26175287, cg27029238, cg27089675, cg27196695, cg27626141

The methods may include a method of analyzing features of the targeted panel from the subject to distinguish between a healthy state and a NAFLD state.

In some cases, the targeted panel includes 5, 6, 7, 8, 9, 10, 11,12,13 or 14 CpG markers selected from the following cgIDs: cg07385778, cg18228076, cg01649623, cg02079413, cg09534872, cg22344162, cg16627358, cg07230621, cg02904344, cg27363529, cg18263455, cg01838971, cg13069385, cg25198847, and cg06012428.

The methods may include a method of analyzing features of the targeted panel from the subject to distinguish between a healthy state and a NASH state. In some cases, the targeted panel includes 5, 6, 7, 8, 9, 10, 11, 12, 13, 14 or 15 CpG markers selected from the following cgIDs: cg06677367, cg01368075, cg05927579, cg13482375, cg00237268, cg16273943, cg16876964, cg00553355, cg23931819, cg05586676, cg07351322, cg23219253, cg12811072, cg00017271, cg11738724, and cg26234543.

The methods may include a method of analyzing features of the targeted panel from the subject to distinguish between a NAFLD state and a NASH state. In some cases, the targeted panel includes 5, 6, 7, 8, 9, 10, 11 or 12 CpG markers selected from the following cgIDs: cg04497820, cg14859874, cg06193597, cg08880261, cg05176970, cg09352518, cg10832239, cg15346191, cg03741619, cg00919702, cg01483656, cg00837987, cg09499109.

The methods may include a method of analyzing features of the targeted panel from the subject to distinguish between a cirrhosis state and a NASH state. In some cases, the targeted panel includes 5, 6, 7, 8, 9, or 10 CpG markers selected from the following cgIDs: cg07475954, cg08844035, cg04682911, cg16822666, cg02376496, cg14861047, cg26123401, cg10284884, cg05959980, cg24005949, cg10180367, cg06733872.

The methods may include a method of analyzing features of the targeted panel from the subject to distinguish between a cirrhosis state and a NAFLD state.

In some cases, the targeted panel includes 5, 6, 7, 8, 9, or 10 CpG markers selected from the following cgIDs: cg10314133, cg22259536, cg11533825, cg04541077, cg04350627, cg23227285, cg16266763, cg09866598, cg25485435, cg20296327, cg10111290.

The methods may include a method of analyzing features of the targeted panel from the subject to distinguish between a healthy obese state and a cirrhosis positive state.

The methods may include a method of analyzing features of the targeted panel from the subject to distinguish between any two of the following: a healthy state; a NAFLD positive state; a NASH positive state; and a cirrhosis positive state.

The methods may include a method of analyzing features of the targeted panel from the subject to distinguish between any two of the following: a healthy state; a NAFLD positive state; a NASH positive state; a cirrhosis positive state; and a liver cancer positive state.

The methods may include a method of analyzing features of the targeted panel from the subject to distinguish between any two of the following: a healthy state; a NAFLD positive state; a NASH positive state; a cirrhosis positive state; and an alcoholic cirrhosis state.

The methods may include a method of analyzing features of the targeted panel from the subject to stage liver fibrosis.

The methods may include a method of analyzing features of the targeted panel from the subject to grade inflammation.

The methods may include a method of analyzing features of the targeted panel from the subject to estimate percent fat in the liver.

In certain embodiments, the diagnosing, staging, or distinguishing has a sensitivity greater than about 50%. In certain embodiments, the diagnosing, staging, or distinguishing has a sensitivity greater than about 75%. In certain embodiments, the diagnosing, staging, or distinguishing has a sensitivity greater than about 90%. In certain embodiments, the diagnosing, staging, or distinguishing has a sensitivity greater than about 99%. In certain embodiments, the diagnosing, staging, or distinguishing has a sensitivity greater than about 99.0%. In certain embodiments, the diagnosing, staging, or distinguishing has a sensitivity approximating 100%.

In certain embodiments, the diagnosing, staging, or distinguishing has a specificity greater than about 50%. In certain embodiments, the diagnosing, staging, or distinguishing has a specificity greater than about 75%. In certain embodiments, the diagnosing, staging, or distinguishing has a specificity greater than about 90%. In certain embodiments, the diagnosing, staging, or distinguishing has a specificity greater than about 99%. In certain embodiments, the diagnosing, staging, or distinguishing has a specificity greater than about 99.0%. In certain embodiments, the diagnosing, staging, or distinguishing has a specificity approximating 100%.

In certain embodiments, the diagnosing, staging, or distinguishing is accomplished without liver biopsy.

The methods may include preparing a sample by a method comprising immunoprecipitation of fragments comprising methylated cytosines.

The methods may include preparing a sample by a method comprising converting unmethylated cytosines to uracils. The conversion may include bisulfite conversion. The conversion may include enzymatic conversion. The enzymatic conversion may include APOBEC-mediated conversion.

The methods may include preparing a sample by a method comprising an amplification step. A set of primers may be selected to amplify DNA encompassing any of the sets of CpG markers. A set of probes may be selected to capture DNA encompassing any of the sets of CpG markers.

### 7 EXAMPLES

### 7.1 Identification of Liver Specific Methylation Markers

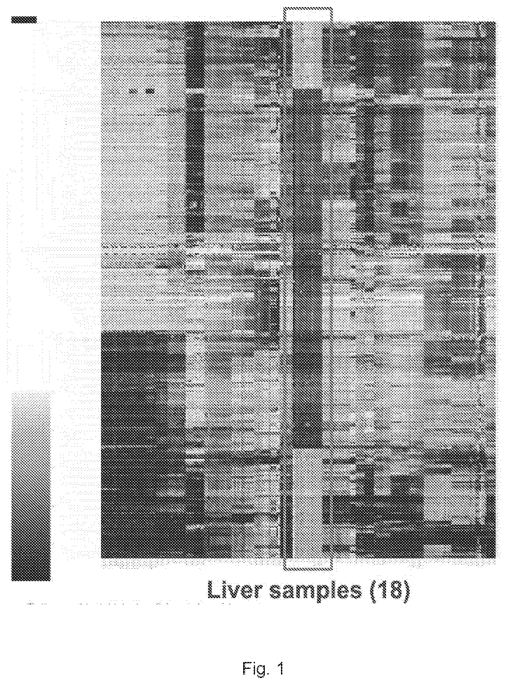

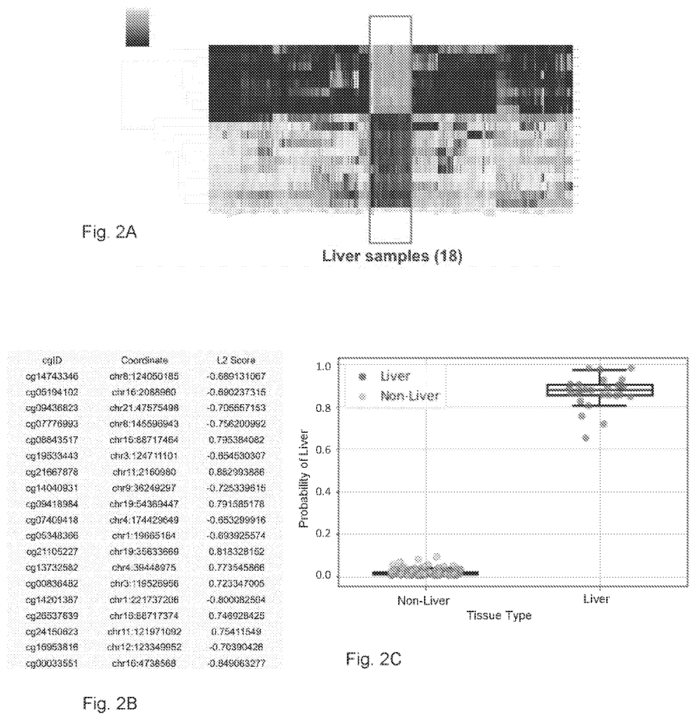

The analysis of liver vs non-liver primary tissue samples for 313 CpGs (FIG. 1) includes samples retrieved from the publicly available databases, the Gene Expression Omnibus (GEO) and The Cancer Genome Atlas (TCGA). The 313 liver-specific CpGs were identified using a dataset composed of blood cell samples (n=36)^{1,2}, brain samples (n=18)^{3,4}, muscle samples (n=10)^{3,5}, heart samples (n=9)^{3,6}, artery/endothelium samples (n=19)^{7,8,9,10,11}, colon samples (n=15)^{12,13,14}, stomach samples (n=12)^{14,15}, esophagus samples (n=6)¹⁴, bladder samples (n=6)¹⁴, spleen samples (n=10)³, fibroblast (n=10)^{16,17}, lung samples (n=15)^{14,18,19}, kidney samples (n=12)^{3,14,20}, pancreas samples (n=19)^{3,11,14} fat samples (n=6)^{3,11} and liver samples (n=18)^{14,11,21,22}. The smaller set of 19 highly predictive liver CpGs (FIG. 2A) uses the same dataset, with the addition of some prostate samples (n=11)^{14,25}.

Tissue-specific CpGs were obtained through multiple rounds of feature selection, the first of which was to select the k most variable CpGs, in order to reduce the number of informative CpGs from around 450,000 CpGs to k=7000. After this, the reduced dataset was analysed using lasso (L1) logistic regression to select features that could discriminate between the chosen disease states. By tweaking the strength of regularization (c parameter) and the number of rounds of L1 feature selection (r), to control the number of predictive CpGs to get many predictive sites. A round of L1 feature selection was run at r=10, and the number of predictive CpGs were controlled, to get 0 rounds with c=0.5. This approach helped identify hundreds of liver-specific CpGs (FIG. 1). By further reducing the number of rounds of L1 feature selection to r=1, a smaller subset of 19 liver-specific markers were gathered. These CpGs were then scored using the coefficients returned from a ridge logistic regression (L2) model in order to evaluate the predictive strength of each marker. After the set of CpGs were established, to evaluate how well these CpGs could discriminate between liver and other tissue samples. In order to do this, the data with only the methylation beta-values, from these 19 CpGs were subsetted, and a cross-validating logistic regression model was trained to evaluate and classify one sample at a time, using all other samples as a training set. This was repeated for all samples within the dataset.

This analysis demonstrates that, using logistic regression, many tissue specific loci (FIG. 1) may be found. Even a small number of CpGs can be highly specific for tissue classification, and can differentiate between liver and non-liver tissue samples with around 90% accuracy (FIG. 2C).

### 7.2 Pairwise Discrimination Between Disease States in Primary Liver Tissues

### 7.2.1 NAFLD Vs Healthy Primary Liver Samples

This analysis of NAFLD vs healthy samples from primary liver tissue included samples retrieved from the publicly available databases the Gene Expression Omnibus (GEO) and The Cancer Genome Atlas (TCGA). Normal liver samples were pulled from GSE48325^{32,33}, GSE78743³, GSE60753³⁴, and TCGA¹⁴ for a total of 57 samples. NAFLD samples were downloaded from GSE48325^{32,33} for a total of 14 samples.

Disease-specific CpGs were obtained through multiple rounds of feature selection, the first of which used mutual information (MI) feature selection to reduce the number of informative CpGs from around 450,000 CpGs to k=1000. After this, the reduced dataset was analysed using lasso (L1) logistic regression to select features that could discriminate between the chosen disease states. By tweaking the strength of regularization (c parameter) and the number of rounds of L1 feature selection (r), the number of predictive CpGs were controlled, to get anywhere around 10 to 20 predictive sites. A number of rounds of L1 feature selection were run at r=5 rounds with c=0.6, Using this approach, a set of 15 CpGs were gathered. These CpGs were then scored using the coefficients returned from a ridge logistic regression (L2) model in order to evaluate the predictive strength of each marker. After the set of CpGs were established, they were evaluated for their accuracy in discriminating between NAFLD and healthy liver samples. In order to do this, the data was subsetted to include only the methylation beta-values from these 15 CpGs, and train a cross-validating logistic regression model to evaluate and classify one sample at a time, using all other samples as a training set. This was repeated for all samples within the dataset.

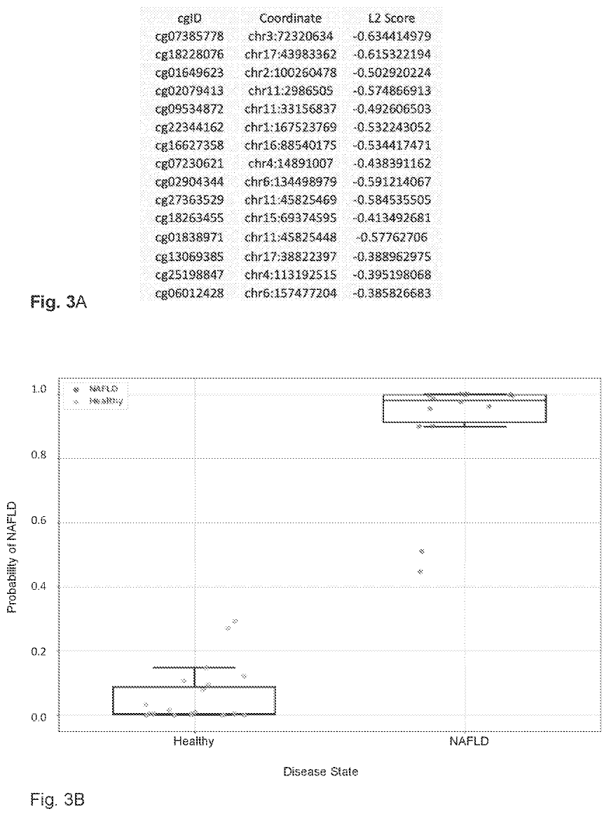

Using only methylation data from the 15 selected CpGs to discriminate between NAFLD and healthy primary liver tissue, each sample was correctly classified as either NAFLD or healthy, with around 90% certainty, confirming the validity of these 15 NAFLD-specific CpGs (FIG. 3B).

### 7.2.2 NASH Vs Healthy Primary Liver Samples

This analysis of NASH vs healthy samples from primary liver tissue included samples retrieved from the publicly available databases the Gene Expression Omnibus (GEO) and The Cancer Genome Atlas (TCGA). Normal liver samples were pulled from GSE48325^{32,33}, GSE78743³, GSE60753³⁴, and TCGA¹⁴ for a total of 57 samples. NASH samples were downloaded from GSE48325^{32,33} for a total of 15 samples.

Disease-specific CpGs were obtained through multiple rounds of feature selection, the first of which used mutual information (MI) feature selection to reduce the number of informative CpGs from around 450,000 CpGs to k=1000. After this, the reduced dataset could then be analysed using lasso (L1) logistic regression to select features that could discriminate between the chosen disease states. By tweaking the strength of regularization (c parameter) and the number of rounds of L1 feature selection (r), the number of predictive CpGs were controlled to get anywhere around 10 to 20 predictive sites. A number of rounds of L1 feature selection were run at r=5 rounds with c=0.6. Using this approach, a set of 16 CpGs were gathered. These CpGs were then scored using the coefficients returned from a ridge logistic regression (L2) model in order to evaluate the predictive strength of each marker. After the set of CpGs were established, the CpGs were evaluated for their accuracy in discriminating between NASH and healthy liver samples. In order to do this, we first subsetted the data to only include methylation beta-values from these 16 CpGs, and trained a cross-validating logistic regression model to evaluate and classify one sample at a time, using all other samples as a training set. This was repeated for all samples within the dataset.

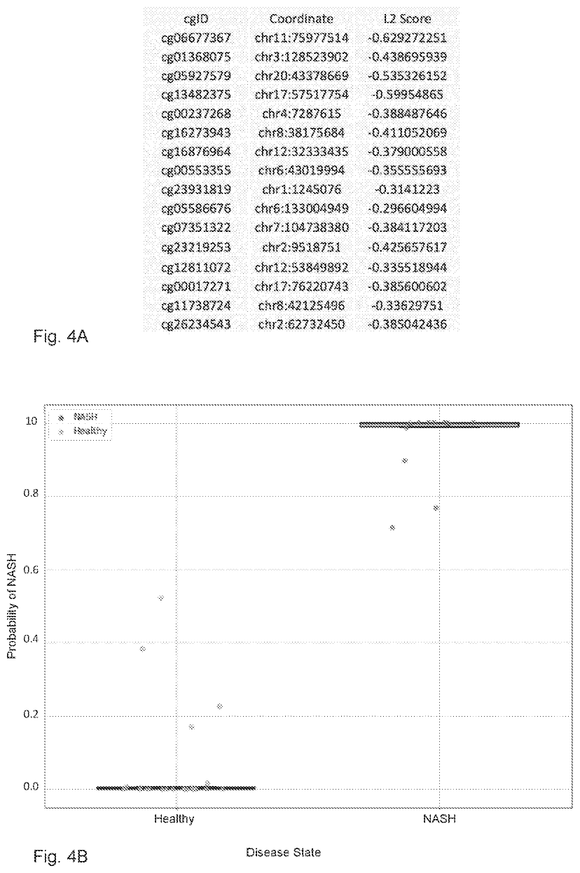

Using only methylation data from the 16 selected CpGs to discriminate between NASH and healthy primary liver tissue, each sample was correctly classified as either NASH or healthy with almost 100% certainty, confirming the validity of these 16 NASH-specific CpGs (FIG. 4B).

### 7.2.3 Cirrhosis Vs Healthy Primary Liver Samples

This analysis of cirrhosis vs healthy samples from primary liver tissue included samples retrieved from the publicly available databases the Gene Expression Omnibus (GEO) and The Cancer Genome Atlas (TCGA). Normal liver samples were pulled from GSE48325^{32,33}, GSE78743³, GSE60753³⁴, and TCGA¹⁴ for a total of 57 samples. Cirrhosis samples were downloaded from GSE60753³⁴ for a total of 77 samples and included the following cirrhotic subtypes: Immune cirrhosis (n=2), genetic cirrhosis (n=4), cryptogenic cirrhosis (n=3), biliary cirrhosis (n=2), ethanol cirrhosis (n=21), HBV cirrhosis (n=6), and HCV cirrhosis (n=39).

Disease-specific CpGs were obtained through multiple rounds of feature selection, the first of which used mutual information (MI) feature selection to reduce the number of informative CpGs from around 450,000 CpGs to k=1000. After this, the reduced dataset could then be analysed using lasso (L1) logistic regression to select features that could discriminate between the chosen disease states. By tweaking the strength of regularization (c parameter) and the number of rounds of L1 feature selection (r), we could control the number of predictive CpGs to get anywhere around 10 to 20 predictive sites. A number of rounds of L1 feature selection were run at r=4 rounds with c=0.5. Using this approach, a set of 20 CpGs were gathered. These CpGs were then scored using the coefficients returned from a ridge logistic regression (L2) model in order to evaluate the predictive strength of each marker. After the set of CpGs were established, these CpGs were evaluated for their accuracy in discriminating between cirrhosis and healthy liver samples. The accuracy of the set of CpGs was evaluated by subsetting the data to include only methylation beta-values from the 20 CpGs, and traine a cross-validating logistic regression model to evaluate and classify one sample at a time, using all other samples as a training set. This was repeated for all samples within the dataset.

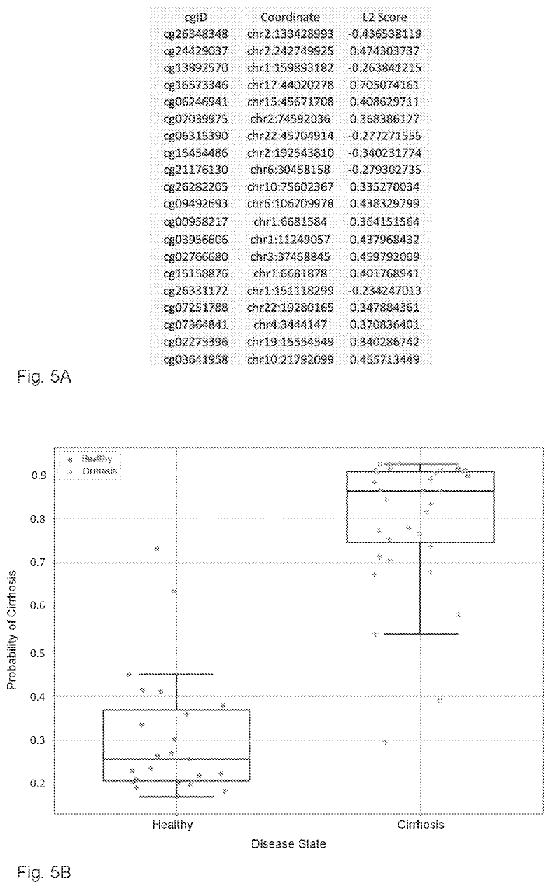

Using only methylation data from the 20 selected CpGs to discriminate between cirrhosis and healthy primary liver tissue, each sample was correctly classified as either cirrhosis or healthy with above 75% certainty, confirming the validity of these 20 cirrhosis-specific CpGs (FIG. 5B). Although these CpGs have predictive certainty that is lower than the NAFLD-specific and NASH-specific CpGs, there is still no statistically significant overlap between the two classified groups. The lower certainty may also be related to the higher variation in types of cirrhotic samples (e.g. ethanol cirrhosis, genetic cirrhosis, immune cirrhosis, etc.), as seen in the materials section.

### 7.2.4 Cirrhosis Vs NAFLD Primary Layer Samples

This analysis of cirrhosis vs NAFLD samples from primary liver tissue included samples retrieved from the Gene Expression Omnibus (GEO), a publicly available database. NAFLD samples were downloaded from GSE48325^{32,33} for a total of 14 samples. Cirrhosis samples were downloaded from GSE60753³⁴ for a total of 77 samples and included the following cirrhotic subtypes: Immune cirrhosis (n=2), genetic cirrhosis (n=4), cryptogenic cirrhosis (n=3), biliary cirrhosis (n=2), ethanol cirrhosis (n=21), HBV cirrhosis (n=6), and HCV cirrhosis (n=39).

Disease-specific CpGs were obtained through multiple rounds of feature selection, the first of which used mutual information (MI) feature selection to reduce the number of informative CpGs from around 450,000 CpGs to k=1000. After this, the reduced dataset could then be analysed using lasso (L1) logistic regression to select features that could discriminate between the chosen disease states. By tweaking the strength of regularization (c parameter) and the number of rounds of L1 feature selection (r), the number of predictive CpGs were controlled to get anywhere around 10 to 20 predictive sites. A number of rounds of L1 feature selection were run at r=4 rounds with c=1.0. Using this approach, a set of 11 CpGs were gathered. These CpGs were then scored using the coefficients returned from a ridge logistic regression (L2) model in order to evaluate the predictive strength of each marker. After the set of CpGs were established, the CpGs were evaluated for their accuracy in discriminating between cirrhosis and NAFLD samples. The accuracy of the set of CpGs was evaluated by subsetting the data to include only methylation beta-values from these 11 CpGs, and traine a cross-validating logistic regression model to evaluate and classify one sample at a time, using all other samples as a training set. This was repeated for all samples within the dataset.

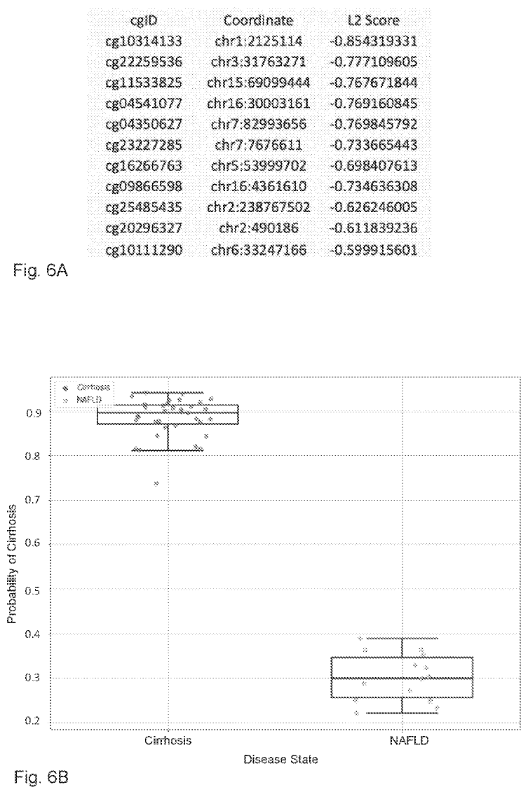

Using only methylation data from the 11 selected CpGs to discriminate between cirrhosis and NAFLD primary liver tissue, each sample was correctly classified as either cirrhosis or NAFLD with an average of around 80% certainty, confirming the validity of these 11 CpGs (FIG. 6). A lower certainty may also be related to the continuous nature of the progression of these liver diseases (i.e., obesity leading to NAFLD, then NASH, cirrhosis, and lastly HCC), and that certain disease states will be harder to distinguish from each other than when they are compared to healthy liver tissues.

### 7.2.5 Cirrhosis Vs NASH Primary Liver Samples

This analysis of cirrhosis vs NASH samples from primary liver tissue included samples retrieved from the Gene Expression Omnibus (GEO), a publicly available database. NASH samples were downloaded from GSE48325^{32,33} for a total of 15 samples. Cirrhosis samples were downloaded from GSE60753³⁴ for a total of 77 samples and included the following cirrhotic subtypes: Immune cirrhosis (n=2), genetic cirrhosis (n=4), cryptogenic cirrhosis (n=3), biliary cirrhosis (n=2), ethanol cirrhosis (n=21), HBV cirrhosis (n=6), and HCV cirrhosis (n=39).

Disease-specific CpGs were obtained through multiple rounds of feature selection, the first of which used mutual information (MI) feature selection to reduce the number of informative CpGs from around 450,000 CpGs to k=1000. After this, the reduced dataset could then be analysed using lasso (L1) logistic regression to select features that could discriminate between the chosen disease states. By tweaking the strength of regularization (c parameter) and the number of rounds of L1 feature selection (r), the number of predictive CpGs were controlled to get anywhere around 10 to 20 predictive sites. A number of rounds of L1 feature selection were run at r=4 rounds with c=1.0, These CpGs were then scored using the coefficients returned from a ridge logistic regression (L2) model in order to evaluate the predictive strength of each marker. After the set of CpGs were established, they were evaluated for their accuracy in discriminating between cirrhosis and NASH samples. In order to do this, the data was subsetted to only include methylation beta-values from these 12 CpGs, and a cross-validating logistic regression model was trained to evaluate and classify one sample at a time, using all other samples as a training set. This was repeated for all samples within the dataset.

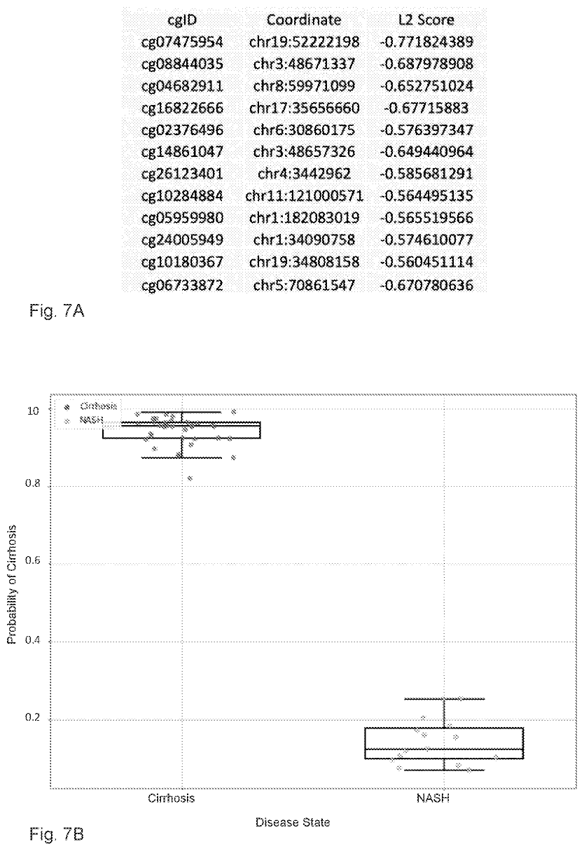

Using only methylation data from the 12 selected CpGs to discriminate between cirrhosis and NAFLD primary liver tissue, each sample was correctly classified as either cirrhosis or NAFLD with an average of around 90% certainty, confirming the validity of these 11 CpGs (FIG. 7B).

### 7.2.6 NAFLD Vs NASH Primary Liver Samples

This analysis of NAFLD vs NASH samples from primary liver tissue included samples retrieved from the Gene Expression Omnibus (GEO), a publicly available database. NAFLD and NASH samples were both downloaded from GSE48325^{32,33} for a total of 14 and 15 samples, respectively

Disease-specific CpGs were obtained through multiple rounds of feature selection, the first of which used mutual information (MI) feature selection to reduce the number of informative CpGs. Due to the high similarity between NAFLD and NASH samples, subject to the continuous nature of liver disease progression as previously described, the MI feature selection was made more liberal than other pairwise comparisons; this ensured the sufficiency of the number of CpGs to select from for the L1 model; from around 450,000 CpGs to k=100000. After this, the reduced dataset was then analysed using lasso (L1) logistic regression to select features that could discriminate between the chosen disease states. By tweaking the strength of regularization (c parameter) and the number of rounds of L1 feature selection (r), the number of predictive CpGs were controlled to get anywhere around 10 to 20 predictive sites. A number of rounds of L1 feature selection were run at r=4, with c=1.0. Using this approach, a set of 13 CpGs were gathered. These CpGs were then scored using the coefficients returned from a ridge logistic regression (L2) model in order to evaluate the predictive strength of each marker. After the set of CpGs were established, these CpGs were evaluated for their accuracy in discriminating between NAFLD and NASH samples. The accuracy of the set of CpGs was evaluated by subsetting the data to include only methylation beta-values from these 13 CpGs, and train a cross-validating logistic regression model to evaluate and classify one sample at a time, using all other samples as a training set. This was repeated for all samples within the dataset.

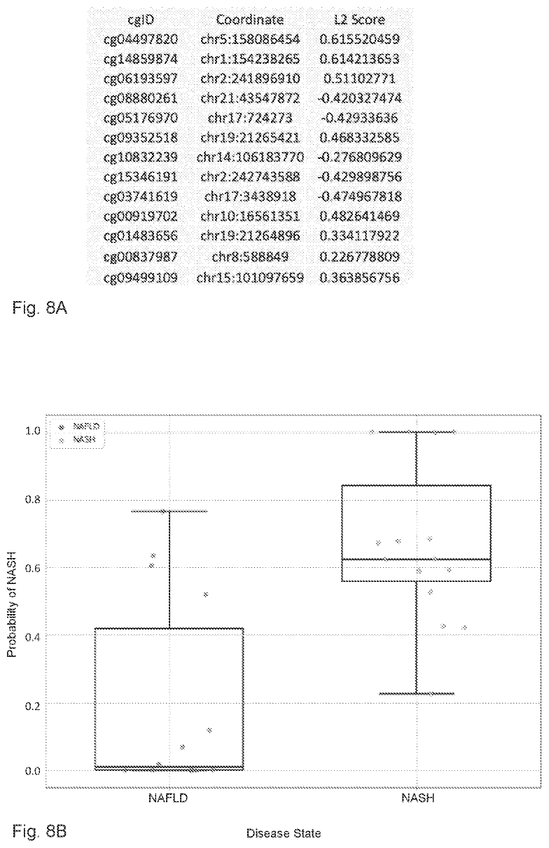

Using only methylation data from the 13 selected CpGs to discriminate between NAFLD and NASH primary liver tissue, each sample was correctly classified as either NAFLD or NASH even with varying degrees of certainty, demonstrating the usefulness of these 13 CpGs (FIG. 8B). As previously mentioned, differentiating between NAFLD and NASH proved to be more difficult as NASH is an extreme case of NAFLD. Their symptoms tend to lie on a continuum, starting with fatigue and abdominal pain in some NAFLD cases, these symptoms also tend to be common with NASH, with severe NASH cases presenting symptoms of cirrhosis and liver failure. Because of these similarities between NAFLD and NASH, a larger degree of variability in the certainty of the sample classifications were expected.

7.3 Pairwise Discrimination Between Liver Disease States in cfDNA Samples
7.3.1 Cirrhosis Vs Healthy cfDNA Samples

This analysis of cirrhosis vs healthy samples from cfDNA included samples retrieved from the Gene Expression Omnibus (GEO), a publicly available database. Normal cfDNA samples were downloaded both from GSE122126¹¹ and GSE110185²⁶, for a total of 14 normal samples. Cirrhotic cfDNA samples were retrieved from GSE12937²⁷, for a total of 44 cirrhotic samples.

Disease-specific CpGs were obtained using a leave-one-out approach, where an individual sample was left out of the dataset for both features selection and model training, followed by the classification of that left-out sample. This ensured that the sample being classified has no influence on how the model selected the features for its classification, and therefore treated the sample as a never seen before patient, as would be the case in a clinical test setting. This entire process was then repeated for each sample in the dataset. The feature selection process used two different approaches in sequence, the first being mutual information (MI) feature selection, which reduced the number of informative CpGs from around 450,000 to k=1,000. The second features selection process used a lasso (L1) logistic regression model to select a smaller number of features that could discriminate between the two disease states. By tweaking the strength of regularization (c parameter) and the number of rounds of L1 feature selection (r parameter), we could control the number of predictive CpGs returned by the model (in this case, we ran r=2 rounds of c=1.0 feature selection to get 6-11 CpGs per left out sample). We then subsetted the data to only these 6-11 selected CpGs, and we trained a ridge (L2) logistic regression (cross-validation) model using all the remaining (n−1) samples. The final left out sample as then classified by the trained model.

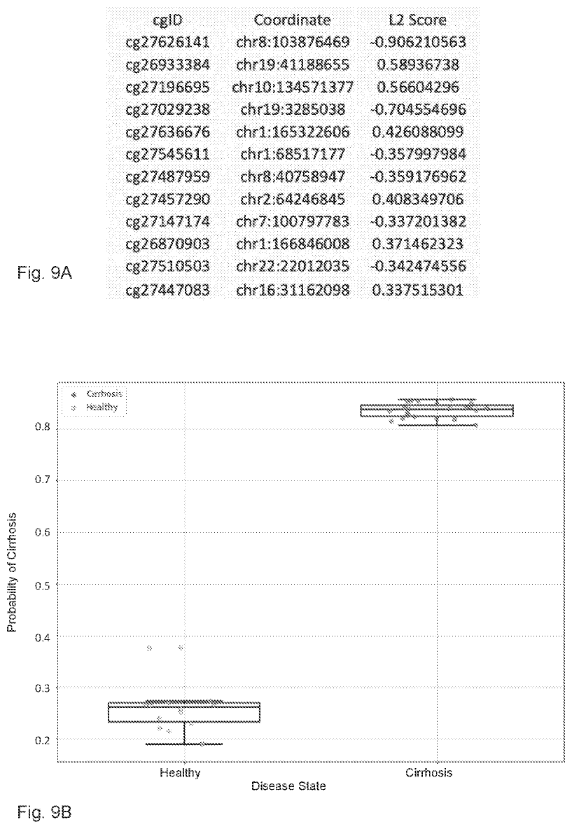

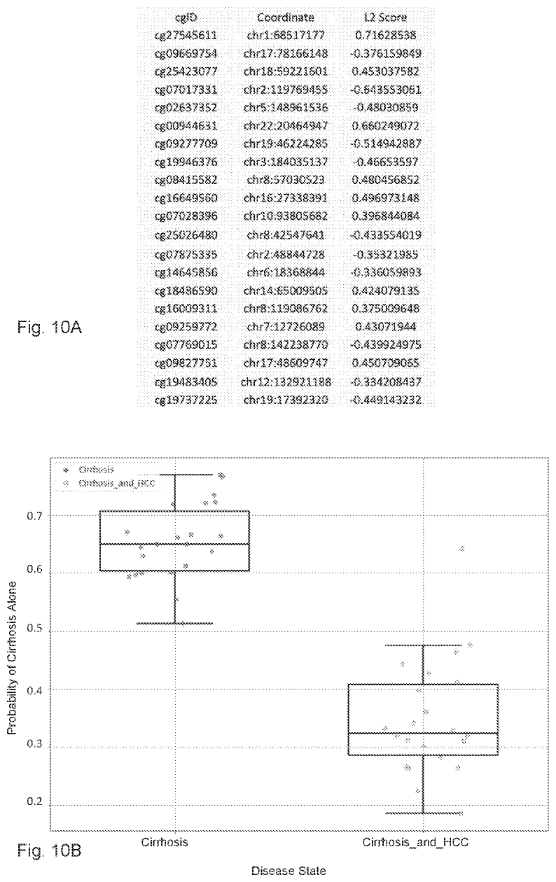

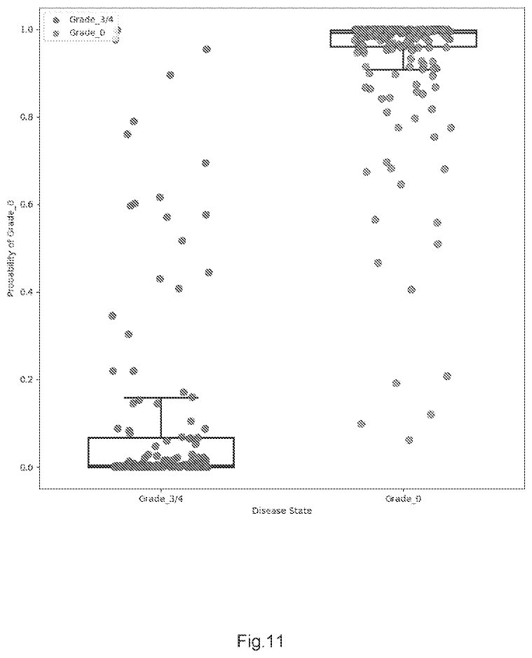

Using this leave-on-out approach, each sample was classified individually as either healthy or cirrhotic (a class that includes samples with cirrhosis or cirrhosis with hepatocellular carcinoma), with a total classification accuracy of 100% (FIG. 9).

### 8 REFERENCES

The entire disclosures of the following references are incorporated into this application by reference.
“Epigenome-wide association study of lung function level and its change” European Respiratory Journal, 2019
Ahrens et al, “DNA methylation analysis in nonalcoholic fatty liver disease suggests distinct disease-specific and remodeling signatures after bariatric surgery” Cell Metabolism, 2013
Ahrens et al, “DNA methylation analysis in nonalcoholic fatty liver disease suggests distinct disease-specific and remodeling signatures after bariatric surgery” Cell Metabolism, 2013
Babikova E A, Generozov E V: Epigenetic analysis of normal prostate tissue and prostate adenocarcinoma. In. Gene Expression Omnibus; 2015
Barberio et al, “Comparison of visceral adipose tissue DNA methylation and gene expression profiles in female adolescents with obesity” Diabetology and Metabolic Syndrome, 2019
Bigot et al, “Age-Associated Methylation Suppresses SPRY1, Leading to a Failure of Re-quiescence and Loss of the Reserve Stem Cell Pool in Elderly Muscle” Cell Reports, 2015
De Geode et al, “Nucleated red blood cells impact DNA methylation and expression analyses of cord blood hematopoietic cells” Clinical Epigenetics, 2015
Díez-Villanueva et al, “DNA methylation events in transcription factors and gene expression changes in colon cancer” Epigenomics, 2020
Gallardo-Gómez, “A new approach to epigenome-wide discovery of non-invasive methylation biomarkers for colorectal cancer screening in circulating cell-free DNA using pooled samples” Clinical Epigenetics, 2018
Hlady et al, “Epigenetic signatures of alcohol abuse and hepatitis infection during human hepatocarcinogenesis” Oncotarget, 2014
Hlady et al, “Genome-wide discovery and validation of diagnostic DNA methylation-based biomarkers for hepatocellular cancer detection in circulating cell free DNA” Theranostics, 2019
Horvath et al, “Obesity accelerates epigenetic aging of human liver” PNAS, 2014
Horvath et al, “The cerebellum ages slowly according to the epigenetic clock” Aging, 2015
Johnson et al, “Differential DNA methylation and changing cell-type proportions as fibrotic stage progresses in NAFLD” Clinical Epigenetics, 2021
Josheph et al, “Epigenome-Wide Association (DNA Methylation) Study of Sex Differences in Normal Human Kidney” Journal of Drug Metabolism and Toxicology, 2017
Kennedy et al, “Critical evaluation of linear regression models for cell-subtype specific methylation signal from mixed blood cell DNA” PLoS One, 2018
Lee et al, “Global DNA Methylation Pattern of Fibroblasts in Idiopathic Pulmonary Fibrosis” DNA and Cell Biology, 2019
Lokk et al, “DNA methylome profiling of human tissues identifies global and tissue-specific methylation patterns” Genome Biology, 2014
Moss et al, “Comprehensive human cell-type methylation atlas reveals origins of circulating cell-free DNA in health and disease” Nature Communications, 2018
Naumov et al, “Genome-scale analysis of DNA methylation in colorectal cancer using Infinium Human Methylation 450 Bead Chips” Epigenetics, 2013
Pervjakova et al, “Imprinted genes and imprinting control regions show predominant intermediate methylation in adult somatic tissues” Epigenomics, 2016
Reinius et al, “Differential DNA methylation in purified human blood cells: implications for cell lineage and studies on disease susceptibility” PLoS One, 2012
Rochtus et al, “Methylome analysis for spina bifida shows SOX18 hypomethylation as a risk factor with evidence for complex (epi)genetic interplay to affect neural tube development” Clinical Epigenetics, 2016
Jung et al, “An LSC epigenetic signature is largely mutation independent and implicates the HOXA cluster in AML pathogenesis” Nature Communications, 2015
TCGA Research Network (https://www.cancer.gov/tcga)
Tobi et al, “DNA methylation as a mediator of the association between prenatal adversity and risk factors for metabolic disease in adulthood” Science Advances, 2018
Valencia-Morales et al, “The DNA methylation drift of the atherosclerotic aorta increases with lesion progression” BMC Medical Genomics, 2015
Vizoso et al, “Aberrant DNA methylation in non-small cell lung cancer-associated fibroblasts” Carcinogenesis, 2015
Wielscher et al, “Diagnositc performance of plasma DNA methylation profiles in lung cancer, pulmonary fibrosis and COPD” EBioMedicine, 2015
Woo et al, “Genome-wide profiling of normal gastric mucosa identifies Helicobacter pylori- and cancer-associated DNA methylome changes” International Journal of Cancer, 2018
Zaina et al, “DNA methylation map of human atherosclerosis” Circulation, 2014
Zhang et al, “The signature of liver cancer in immune cells DNA methylation” Clinical Epigenetics, 2018
Zhou et al, “Human atrium transcript analysis of permanent atrial fibrillation” International Heart Journal, 2014
Zhu et al, “Whole-genome transcription and DNA methylation analysis of peripheral blood mononuclear cells identified aberrant gene regulation pathways in systemic lupus erythematosus” Arthritis Research and Therapy, 2016

We claim:

1. A method of classifying a liver disease by analyzing a DNA sample, wherein the DNA sample comprises cfDNA and/or blood cell DNA, the method comprising:
(a) obtaining the DNA sample;
(b) determining CpG methylation status at CpG sites of DNA molecules of the DNA sample;
(c) identifying a methylation pattern based on the CpG methylation status of the DNA molecules;
(d) assigning to the DNA sample a non-cancer liver disease classification based on the methylation pattern.

2. The method of claim 1, wherein the DNA sample comprises fragments, wherein the fragments comprise cfDNA fragments or DNA fragments from blood cells.

3. The method of claim 2, further comprising performing shearing or restriction digestion on the fragments to obtain smaller-sized fragments.

4. The method of claim 2, further comprising enriching the fragments by hybridization to a set of probes of a targeted panel or by performing PCR with a panel of primers.

5. The method of claim 1, further comprising calculating, using the methylation pattern, a methylation level indicating a probability that the DNA sample belongs to the liver disease classification.

6. The method of claim 5, wherein (d) comprises comparing the methylation level to a cut-off to classify the liver disease with the liver disease classification.

7. The method of claim 1, wherein (d) comprises classifying the DNA sample as having a probability of:
(a) no liver disease;
(b) non-alcoholic fatty liver disease;
(c) non-alcoholic steatohepatitis; or
(d) liver cirrhosis.

8. The method of claim 1, wherein (d) comprises classifying the DNA sample for a stage of fibrosis.

9. The method of claim 8, wherein the classifying the DNA sample for the stage of fibrosis comprises classifying the DNA sample as having a probability of:
(a) no fibrosis;
(b) portal fibrosis without septa;
(c) portal fibrosis with few septa;
(d) periportal fibrosis;
(e) bridging fibrosis;
(f) F0 fibrosis;
(g) F1 fibrosis;
(h) F2 fibrosis;
(i) F3 fibrosis;
(j) F4 fibrosis; or
(k) cirrhosis.

10. The method of claim 1, wherein (d) comprises classifying the DNA sample for having a probability of:
(a) no hepatitis;
(b) non-specific reactive hepatitis;
(c) granulomatous hepatitis;
(d) chronic active hepatitis;
(e) acute hepatitis;
(f) autoimmune hepatitis;
(g) alcoholic hepatitis; or
(h) nonalcoholic hepatitis.

11. The method of claim 1, wherein (d) comprises classifying the DNA sample for having a probability of:
(a) no inflammation;
(b) mild inflammation;
(c) moderate inflammation; or
(d) marked or severe inflammation.

12. The method of claim 1, wherein (d) comprises classifying the DNA sample for having a probability of:
(a) no necrosis;
(b) mild necrosis;
(c) moderate necrosis; or
(d) marked or severe necrosis.

13. The method of claim 1, wherein (d) comprises classifying the DNA sample for a level of fat in the liver.

14. The method of claim 1, further comprising reporting a probability of a stage of the liver disease with a score derived from a methylation level of the DNA sample.

15. The method of claim 14, wherein the methylation level is established by identifying one or more coefficients for one or more CpG features by fitting a model based on the methylation pattern in the DNA sample.

16. The method of claim 15, wherein the model is fitted using a training data set comprising DNA samples from:
(a) subjects with the liver disease; and
(b) subjects without the liver disease.

17. The method of claim 15, wherein the one or more CpG features comprise a single CpG site.

18. The method of claim 15, wherein the one or more CpG features are derived using:
(a) mutual information analysis; or
(b) L1 logistic regression.

19. The method of claim 15, wherein the model comprises:
(a) a logistic regression model;
(b) a logistic regression model with L2 penalty;
(c) a logistic regression model with L1 penalty;
(d) a random forest model;
(e) a neural network model;
(f) a support vector machine;
(g) a gradient boosting algorithm; or
(h) a naive Bayes algorithm.

20. The method of claim 1, wherein the DNA sample comprises genomic regions that are enriched by a targeted panel, wherein the panel is established by a method comprising:
(a) selecting a set of genomic regions based on cfDNA samples from subjects with and without the liver disease, using:
(i) mutual information;
(ii) variation based on a cutoff requirement; or
(iii) L1 logistic regression; and
(b) selecting a set of genomic regions based on liver tissue DNA samples from subjects with and without the liver disease, using:
(i) mutual information;
(ii) variation based on a cutoff requirement; or
(iii) L1 logistic regression; and
(c) selecting a set of genomic regions based on samples of DNA obtained from purified hepatocytes, adipocytes, fibroblasts, and/or immune cells using:
(i) mutual information;
(ii) variation based on a cutoff requirement; or
(iii) L1 logistic regression.

21. The method of claim 1, wherein the DNA sample comprises DNA from a blood cell sample, and wherein the DNA from the blood cell sample comprises genomic regions that are enriched by a targeted panel, which is established by a method comprising:
(a) selecting a set of genomic regions based on blood cell samples from a training set from subjects with and without the liver disease using:
(i) mutual information;
(ii) variation based on a cutoff requirement; or
(iii) L1 logistic regression; and
(b) selecting a set of genomic regions based on samples from purified T cells, B cells, granulocytes and/or neutrophils using:
(i) mutual information;
(ii) variation based on a cutoff requirement; or
(iii) L1 logistic regression.

22. The method of claim 1, wherein (b) comprises determining the presence of 5mC or 5hmC modifications at individual sites of the DNA molecules using methylation-aware sequencing.

23. The method of claim 1, wherein (b) comprises determining average levels of 5mC or 5hmC across individual genomic CpG sites of the DNA molecules using a methylation-aware DNA array method, PCR, qPCR or digital PCR.

24. The method of claim 1, wherein (b) comprises converting the DNA molecules using (i) sodium bisulfite treatment, or (ii) TET2-assisted DNA oxidation and APOBEC-assisted cytosine deamination.

25. The method of claim 1, wherein (b) comprises binding the DNA molecules to a DNA array and enriching the DNA sample using probes from a targeted panel.

26. The method of claim 1, wherein (b) comprises performing methylation-aware sequencing of the DNA molecules.

27. The method of claim 1, wherein (b) comprises detecting methylation levels of the CpG sites of the DNA molecules using a DNA array, PCR, qPCR or digital PCR.

28. The method of claim 1, further comprising administering to a subject a therapy selected to treat a disease corresponding to the liver disease classification.

29. A method of classifying a liver disease by analyzing a DNA sample, wherein the DNA sample comprises cfDNA and/or blood cell DNA, the method comprising:
(a) obtaining the DNA sample;
(b) determining CpG methylation status at CpG sites of DNA molecules of the DNA sample;
(c) identifying a methylation pattern based on the CpG methylation status of the DNA molecules;
(d) assigning to the DNA sample a liver disease classification based on the methylation pattern, wherein the liver disease classification distinguishes between (i) a liver cancer positive state and (ii) a liver disease state that progresses into the liver cancer positive state.

30. A method of classifying a liver disease by analyzing a DNA sample, wherein the DNA sample comprises cfDNA and/or blood cell DNA, the method comprising:
(a) obtaining the DNA sample;
(b) determining CpG methylation status at CpG sites of DNA molecules of the DNA sample;
(c) identifying a methylation pattern based on the CpG methylation status of the DNA molecules;
(d) assigning to the DNA sample a liver disease classification based on the methylation pattern, wherein the liver disease classification distinguishes between (i) a healthy state, (ii) a NAFLD positive state, (iii) a NASH positive state, and (iv) a cirrhosis positive state.
